(19) United States
(12) Patent Application Publication
(10) Pub. No.: US 2012/0197244 A1
(43) Pub. Date: Aug. 2, 2012

(54) TREATMENT FOR ANEURYSM

(75) Inventors: Robert J. Siegel, Beverly Hills, CA (US); Michael Shehata, Manhattan Beach, CA (US)

(73) Assignee: CEDARS-SINAI MEDICAL CENTER, Los Angeles, CA (US)

(21) Appl. No.: 13/361,576

(22) Filed: Jan. 30, 2012

Related U.S. Application Data
(60) Provisional application No. 61/480,265, filed on Apr. 28, 2011.
(60) Provisional application No. 61/438,137, filed on Jan. 31, 2011.

(51) Int. Cl. A61B 18/04; A61B 18/18; A61B 18/12; A61B 18/02

(52) U.S. Cl. 606/21; 606/28; 606/33; 606/41

(57) ABSTRACT

The invention relates to methods for treating, inhibiting, reducing the size of or preventing enlargement of, aneurysms in a subject by delivering thermal or cryo energy to the vasculature of the subject at or near the site of an existing or potential aneurysm.

### CROSS-REFERENCE TO RELATED APPLICATIONS

This application claims priority to U.S. Ser. No. 61/438,137 filed Jan. 31, 2011 and to U.S. Ser. No. 61/480,265 filed Apr. 28, 2011, the contents of all of which are herein incorporated by reference.

### FIELD OF INVENTION

The invention relates to the treatment of aneurysms using temperature adjustment at or near the site of aneurysm.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

An aneurysm is an abnormal stretching of the wall of the artery or vein, typically, about 1.5-2 times the normal diameter. Aneurysms can commonly occur in arteries at the base of the brain; an aortic aneurysm occurs in the main artery carrying blood from the left ventricle of the heart. When the size of an aneurysm increases, there is a significant risk of rupture, resulting in severe hemorrhage, other complications or even death. Aneurysms can be hereditary or caused by disease such as high blood pressure, both of which cause the wall of the blood vessel to weaken.

Current treatments for arterial aneurysms are expensive, invasive, high-risk and often associated with prolonged hospitalization and recovery. There is a need for safer and less invasive treatments for arterial aneurysms.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

The present invention is directed to methods for treating aneurysms—arterial or otherwise—in a subject in need thereof comprising providing a catheter having an energy transfer and/or temperature-adjusting element, placing the catheter at or near the site of the aneurysm and delivering thermal or cryo energy to the vasculature in the subject, thereby treating the aneurysm in the subject.

The present invention also provides methods for inhibiting aneurysms—arterial or otherwise—in a subject in need thereof comprising providing a catheter having an energy transfer and/or temperature-adjusting element, placing a catheter at or near the site of the potential or existing aneurysm and delivering thermal or cryo energy to the vasculature in the subject, thereby inhibiting the aneurysm in the subject.

The invention further provides methods for reducing aneurysms—arterial or otherwise—in a subject in need thereof comprising providing a catheter having an energy transfer and/or temperature-adjusting element, placing a catheter at or near the site of the aneurysm and delivering thermal or cryo energy to the vasculature in the subject, thereby treating the aneurysm in the subject.

The instant invention is also directed to methods for preventing enlargement of aneurysms—arterial or otherwise—in a subject in need thereof comprising providing a catheter having an energy transfer and/or temperature-adjusting element, placing a catheter at the site of the aneurysm and delivering thermal or cryo energy to the vasculature in the subject, thereby treating arterial aneurysm in the subject.

In one embodiment of the invention, the vasculature in the subject is the artery, vein or aorta in the subject. In a further embodiment, the arterial aneurysm is a saccular aneurysm. In another embodiment, the arterial aneurysm is a fusiform aneurysm, including but not limited to fusiform aneurysm of the aortic root, ascending aorta, aortic arch, descending aorta and/or abdominal aorta. The aneurysm in the abdominal aorta may be infrarenal or suprarenal aneurysms. In a further embodiment of the invention, the arterial aneurysm is a peripheral aneurysm.

In an embodiment of the invention, thermal energy may be delivered using high intensity frequency ultrasound (HIFU). In some embodiments, the effective frequency for HIFU delivery for use with the claimed methods may be in the range of 1 to 50 MHz, 0.5 to 5 MHz, 5 to 10 MHz, 10-15 MHz, 15-20 MHz, 20-25 MHz, 25-30 MHz, 30-35 MHz, 35-40 MHz, 40-45 MHz, 45-50 MHz, 1 to 40 MHz, 1 to 30 MHz, 1 to 20 MHz, 1 to 10 MHz or 1 to 5 MHz.

In various other embodiments, thermal energy may be delivered to the vasculature using various other methods including but not limited to electric energy, micro-wave energy, radio-frequency energy, magnetic resonance energy and/or X-Ray energy. The electric energy may be in the form of a direct current or pulsating direct current, or a combination thereof. The electrical energy may be in the form of direct or alternating current or a combination thereof.

In another embodiment, cryo energy may be delivered at or near the site of an existing or potential aneurysm by use of cryoablation or other mechanism for lowering the temperature to a state that is significantly colder than normal body temperature. In some embodiments, the effective range of temperature may include freezing the tissue to a temperature of about −40° Celsius, −45° Celsius, −50° Celsius, −55° Celsius, −60° Celsius, −65° Celsius, −70° Celsius, −75° Celsius or −80° Celsius. In other embodiments, the lowered temperature is applied for a time of about 0.5 minutes, 1.0 minutes, 1.5 minutes, 2.0 minutes, 2.5 minutes, 3.0 minutes, 3.5 minutes or 4.0 minutes. Although actual or potential aneurysms in any number of blood vessels may be addressed through this embodiment of the invention, it may be especially useful in the ascending or descending aorta.

In an embodiment of the invention, the catheter is a balloon catheter, for example a high intensity focused ultrasound catheter with a balloon. In another embodiment, the catheter is without a balloon. The catheters may be any size. One skilled in the art can readily determine catheter sizes that are suitable for the claimed methods, for example in a human subject. In one embodiment, catheter sizes 5 to 14 French, inclusive, may be particularly useful.

There are many devices readily available to practice the claimed invention. These devices include but are not limited to HIFU balloon catheters from ProRhythm, Inc. or ReCor Inc.; balloon silicone catheters from Dow Corning, Sonablate® 500 from Misonix, Inc. or Ablatherm® from EDAP TMS S.A; and cryoablation catheters from Medtronic, Inc., Arctic Front Cryoballon® or Freezor Xtra/Max® ablation catheters.

An embodiment of the claimed methods provides that the balloon catheter is placed percutaneously at the site of the blood vessel dilation or aneurysm. High intensity focused ultrasound is delivered at a frequency from 1 to 30 MHz. The acoustic power output of this system may range from 1 to 90 watts. Various embodiments of the invention utilize high intensity frequency ultrasound which can be guided by transcutaneous or intravascular ultrasound, magnetic resonance imaging, computerized tomographic imaging or routine radiographic X-ray imaging with our without contrast, including fluoroscopy. This percutaneous and/or transcutaneous therapy of arteraila or venous segments is intented to cause shrinkage and to reduce and/or stabilize arterail aortic or venous aneurysms or ectasias.

In a further embodiment, thermal or cryo energy may be delivered in an intra-luminal or extra-luminal fashion, circumferentially, perpendicularly or applied in a series of arcs encompassing 360 degree circumference of the blood vessel. Thermal or cryo energy may also be applied sequentially or in a single application perpendicularly through a catheter shaft to a vascular segment which may include a segment of the aorta, peripheral arterial segments as well as venous ectasias. The energy may be delivered in a single pulse for 1 second up to 360 seconds or in multiple pulses ranging from 1 to 100 pulses at time intervals from 1 to 360 seconds, with the intent to produce heating without significant vascular damage.

In an embodiment of the invention, thermal or cryo energy delivery device utilize high intensity ultrasound which can be guided by transcutaneous or intravascular ultrasound or magnetic resonance imaging or computerized tomographic imaging or routine radiographic X-ray imaging with our without contrast, including fluoroscopy. This percutaneous and/or transcutaneous therapy of arteraila or venous segments is intented to cause shrinkage and to reduce and/or stabilize arterail aortic or venous aneurysms or ectasias.

In a further embodiment, cryo energy may be delivered in an intra-luminal or extra-luminal fashion, circumferentially, perpendicularly or applied in a series of arcs encompassing 360 degree circumference of the blood vessel. Cryo energy may also be applied sequentially or in a single application perpendicularly through a catheter shaft to a vascular segment which may include a segment of the aorta, peripheral arterial segments as well as venous ectasias. The energy may be delivered in a single pulse or a series of pulses lasting for 1 second up to 4 minutes each.

The subjects treated by the present invention include mammalian subjects, including, human, monkey, ape, dog, cat, cow, horse, goat, pig, rabbit, mouse and rat.

The various methods of the invention may be performed in any number of contexts as will be readily appreciated by those of skill in the art, such as, but in no way limited to, intra-operatively.

### Advantages of the Invention

The current treatments for aneurysm are open surgical repair and in certain instances aneurysms in the descending or abdominal aorta may be repaired by transcetheter stent grafting. The instant invention is less invasive, less traumatic and may be applied to situations not suitable for stent grafting such as the aortic root or ascending aorta. Additionally, since the claimed methods are minimally invasive and very low risk, aneurysms may be treated earlier.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed:

1. A method for treating an aneurysm, inhibiting an aneurysm, reducing an aneurysm or preventing enlargement of an aneurysm in a subject in need thereof comprising:
(i) providing a catheter having an energy transfer and/or temperature-adjusting element;
(ii) placing the catheter at or near the site of the aneurysm; and
(iii) delivering thermal or cryo energy to the vasculature in the subject using the catheter, thereby treating the aneurysm, inhibiting the aneurysm, reducing the aneurysm or preventing enlargement of the aneurysm in the subject.

2. The method of claim 1, wherein the thermal energy is delivered using high intensity focused ultrasound (HIFU).

3. The method of claim 2, wherein HIFU is delivered at a frequency of 1 to 30 MHz.

4. The method of claim 1, wherein the vasculature is an artery, vein or an aorta.

5. The method of claim 1, wherein the catheter is a balloon catheter.

6. The method of claim 1, wherein the aneurysm is a saccular aneurysm.

7. The method of claim 1, wherein the aneurysm is a fusiform aneurysm.

8. The method of claim 1, wherein the aneurysm is peripheral arterial aneurysm.

9. The method of claim 7, wherein the fusiform aneurysm is of the aortic root, ascending aorta, aortic arch, descending aorta and abdominal aorta.

10. The method of claim 1, wherein the thermal or cryo energy is from any one or more of electrical energy, microwave energy, radio-frequency energy, magnetic resonance energy or X-ray energy.

11. The method of claim 1, wherein the thermal or cryo energy is delivered in a single pulse for 1 second up to 360 seconds.

12. The method of claim 1, wherein the thermal or cryo energy is delivered in multiple pulses ranging from 1 to 100 pulses at time intervals from 1 to 360 seconds.

13. The method of claim 1, wherein the catheter is placed percutaneously.

14. The method of claim 1, wherein the catheter is placed transcutaenously.

15. The method of claim 1, wherein the thermal or cryo energy is applied intra-luminally, extra-luminally, circumferentially, perpendicularly, or applied in a series of arcs encompassing 360 degree circumference of a blood vessel.

16. The method of claim 1, wherein the temperature is adjusted by being lowered to a temperature substantially lower than normal body temperature with the delivery of cryo energy.
